(19) United States
(12) Patent Application Publication
(10) Pub. No.: US 2022/0134050 A1
(43) Pub. Date: May 5, 2022

(54) ENVIRONMENT CONTROL SYSTEM AND METHOD FOR CONTROLLING ENVIRONMENT

(71) Applicant: Mitsubishi Electric Corporation, Tokyo (JP)

(72) Inventor: Hiroshi MORIYASU, Tokyo (JP)

(21) Appl. No.: 17/433,314

(22) PCT Filed: Apr. 25, 2019
(86) PCT No.: PCT/JP2019/017640

§ 371 Date: Aug. 24, 2021

(51) Int. Cl. A61M 21/02; F24F 11/66

(52) U.S. Cl. CPC A61M 21/02; F24F 11/66

(57) ABSTRACT

An environment control system includes: an environment control apparatus installed in a room; a human body state detection unit configured to detect human body sensor information; an environmental state detection unit configured to detect environmental sensor information; a sleep state determination unit configured to determine whether a sleep state is any one of a hypnagogic state, a sleeping state or a wakefulness state; a learning unit configured to learn a predicted sleep rhythm of the human body based on the human body sensor information, the environmental sensor information, and a determination result of the sleep state; a control content determination unit configured to determine control content of the environment control apparatus such that the predicted sleep rhythm obtained by learning approaches an ideal sleep rhythm; and an apparatus control unit configured to control the environment control apparatus based on the control content determined.

### TECHNICAL FIELD

The present disclosure relates to an environment control system including an environment control apparatus that adjusts an indoor environment and to a method for controlling an environment.

### BACKGROUND ART

In recent years, it has been pointed out that the aging of the population, the diversification of lifestyles, disturbance of circadian rhythms, and stress, for example, reduce sleeping comfort. If a reduction in sleeping comfort causes sleeping disorders, daily life or social life may be interfered with due to daytime sleepiness, fatigue, or loss of concentration. Therefore, there has been a demand for improvement in sleeping comfort from the viewpoint of enhancement of productivity and health management in a company, for example.

It is known that sleeping comfort significantly changes corresponding to a sleep environment, such as a temperature, humidity, illuminance, and noises during sleep. In view of the above, recently, various systems that improve a sleep environment have been proposed to enhance sleeping comfort.

For example, Patent Literature 1 discloses an air conditioning system that enhances sleep quality during sleep. In this air conditioning system, based on a database where detection results from a temperature sensor and a humidity sensor are associated with various time periods relating to sleep quality, such as a time period of wakefulness and a time period of REM sleep, a target temperature and target humidity for an air-conditioning apparatus are set such that the best sleep quality can be acquired.

### CITATION LIST

### Patent Literature

Patent Literature 1: Japanese Patent No. 5309742

### SUMMARY OF INVENTION

### Technical Problem

It is known that sleep has sleep rhythm where shallow sleep and deep sleep are repeated. Sleep has suitable environmental states corresponding to the depth of sleep. By appropriately maintaining the environmental state corresponding to a sleep state, it is possible to achieve sleep with an ideal sleep rhythm.

However, in the system described in Patent Literature 1, the target temperature and target humidity during sleep for the air-conditioning apparatus are set to constant values and hence, the temperature and humidity are adjusted such that a constant environmental state is maintained. That is, it is difficult for this system to provide an appropriate environmental state corresponding to the depth of sleep.

The present disclosure has been made in view of a problem of the above-mentioned conventional technique, and it is an object of the present disclosure to provide an environment control system and a method for controlling an environment that can achieve a sleep environment where rhythm during sleep assumes an appropriate sleep rhythm.

### Solution to Problem

An environment control system of one embodiment of the present disclosure includes: an environment control apparatus installed in a room, and configured to adjust an indoor environment; a human body state detection unit configured to detect a state of a human body present in the room as human body sensor information; an environmental state detection unit configured to detect an environmental state in the room as environmental sensor information; a sleep state determination unit configured to determine, based on the human body sensor information, whether a sleep state is any one of a hypnagogic state, which is a state before the human body falls asleep, a sleeping state where the human body is asleep, or a wakefulness state where the human body is in a transitional state from sleep to being awake; a learning unit configured to learn a predicted sleep rhythm of the human body based on the human body sensor information, the environmental sensor information, and a determination result of the sleep state; a control content determination unit configured to determine control content of the environment control apparatus such that the predicted sleep rhythm obtained by learning approaches an ideal sleep rhythm; and an apparatus control unit configured to control the environment control apparatus based on the control content determined.

A method for controlling an environment of another embodiment of the present disclosure includes the steps of: detecting a state of a human body present in a room as human body sensor information; detecting an environmental state in the room as environmental sensor information; determining, based on the human body sensor information, whether a sleep state is any one of a hypnagogic state, which is a state before the human body falls asleep, a sleeping state where the human body is asleep, or a wakefulness state where the human body is in a transitional state from sleep to being awake; learning a predicted sleep rhythm of the human body based on the human body sensor information, the environmental sensor information, and a determination result of the sleep state; deciding control content of an environment control apparatus such that the predicted sleep rhythm obtained by learning approaches an ideal sleep rhythm, the environment control apparatus being installed in the room; and controlling the environment control apparatus based on the control content determined.

### Advantageous Effects of Invention

As described above, according to the embodiments of the present disclosure, the environment control apparatus 40 is controlled such that the predicted sleep rhythm, which is learned based on human body sensor information and environmental sensor information, approaches the ideal sleep rhythm. Therefore, it is possible to achieve a sleep environment where rhythm during sleep assumes an appropriate sleep rhythm.

### BRIEF DESCRIPTION OF DRAWINGS

FIG. 1 is a hardware configuration diagram showing one example of the configuration of an environment control system according to Embodiment 1.

FIG. 2 is a block diagram showing one example of the configuration of a controller shown in FIG. 1.

FIG. 3 is a schematic view showing one example of an ideal sleep rhythm.

FIG. 4 is a schematic view showing one example of a bad sleep rhythm.

FIG. 5 is a flowchart showing one example of the flow of sleep environment improving processing performed by the environment control system according to Embodiment 1.

FIG. 6 is a hardware configuration diagram showing one example of the configuration of an environment control system according to Embodiment 2.

FIG. 7 is a block diagram showing one example of the configuration of a controller shown in FIG. 6.

FIG. 8 is a hardware configuration diagram showing one example of the configuration of an environment control system according to Embodiment 3.

FIG. 9 is a block diagram showing one example of the configuration of a controller shown in FIG. 8.

FIG. 10 is a hardware configuration diagram showing one example of the configuration of an environment control system according to Embodiment 4.

FIG. 11 is a block diagram showing one example of the configuration of a controller shown in FIG. 10.

FIG. 12 is a hardware configuration diagram showing one example of the configuration of an environment control system according to Embodiment 5.

FIG. 13 is a block diagram showing one example of the configuration of a controller shown in FIG. 12.

FIG. 14 is a hardware configuration diagram showing one example of the configuration of an environment control system according to Embodiment 6.

FIG. 15 is a block diagram showing one example of the configuration of a controller shown in FIG. 14.

### DESCRIPTION OF EMBODIMENTS

Hereinafter, Embodiments of the present disclosure will be described with reference to drawings. In the following drawings, identical or corresponding components are given the same reference symbols, and the repeated description of such components will be omitted. Modes of constitutional elements described in entire Description are merely for the sake of example. The present disclosure is not limited to the constitutional elements described in Description.

### Embodiment 1

Hereinafter, an environment control system according to Embodiment 1 will be described. The environment control system according to Embodiment 1 is configured such that various apparatuses forming the system are provided in a room, such as a bedroom, to adjust an indoor environment corresponding to the sleep state of the human body.

### [Configuration of Environment Control System 100]

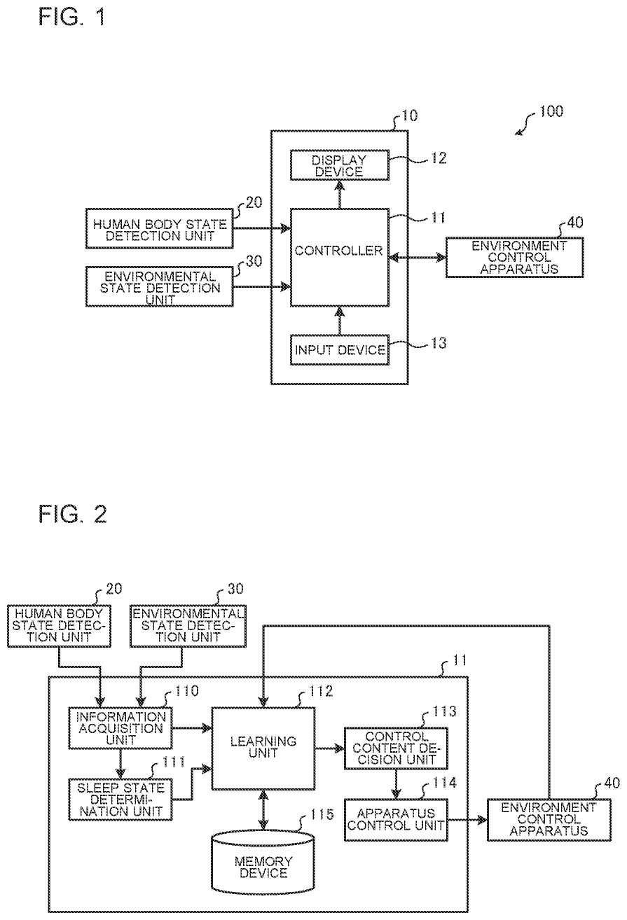

FIG. 1 is a hardware configuration diagram showing one example of the configuration of an environment control system 100 according to Embodiment 1. As shown in FIG. 1, the environment control system 100 includes a management device 10, a human body state detection unit 20, an environmental state detection unit 30, and an environment control apparatus 40. In the environment control system 100, the human body state detection unit 20, the environmental state detection unit 30, and one or a plurality of environment control apparatuses 40 are connected to the management device 10 by wired or wireless communication.

The human body state detection unit 20 detects the state of the human body during sleep, and outputs human body sensor information. The human body state detection unit 20 is a human body sensor that detects physical quantities relating to the human body, such as a body temperature, body movements and heartbeat, and outputs the physical quantities as human body sensor information. In Embodiment 1, a period of hypnagogia, sleeping and wakefulness is collectively referred to as “sleep”.

The environmental state detection unit 30 detects a state in the room, and outputs environmental sensor information. The environmental state detection unit 30 is an environmental sensor that detects physical quantities relating to an environment, such as a temperature, humidity and illuminance, and outputs the physical quantities as environmental sensor information.

The environment control apparatus 40 is an apparatus for adjusting an environment in the room. For the environment control apparatus 40, for example, an air-conditioning device, a lighting device, or an audio apparatus may be used. In Embodiment 1, the environment control apparatus 40 controls, for example, a temperature and humidity, light, and sound in the room during sleep.

The management device 10 manages the environment in the room based on the human body state detection unit 20 and the environmental state detection unit 30, and controls the action of the environment control apparatus 40. As shown in FIG. 1, the management device 10 includes a controller 11, a display device 12, and an input device 13.

The display device 12 displays an environmental state in the room and the sleep state of the human body, for example. The display device 12 may be a liquid crystal display (LCD) or an organic electro luminescence (EL) display, for example. It is also possible to use, as the display device 12, a touch panel display where a touch panel including touch sensors is stacked on an LCD or an organic EL display, for example.

The input device 13 is used to operate the management device 10, and outputs operation signals corresponding to operations performed by the user. For example, keys or the like provided to the management device 10 are used as the input device 13. In the case where the display device 12 is a touch panel display, various keys may be displayed on the display device 12 as software keys. In the management device 10, the display device 12 and the input device 13 are not essential components, and may be provided when necessary.

Based on human body sensor information detected by the human body state detection unit 20 and environmental sensor information detected by the environmental state detection unit 30, the controller 11 controls the environment control apparatus 40 such that the human body enters a comfortable sleep state. FIG. 2 is a block diagram showing one example of the configuration of the controller 11 shown in FIG. 1. As shown in FIG. 2, the controller 11 includes an information acquisition unit 110, a sleep state determination unit 111, a learning unit 112, a control content determination unit 113, an apparatus control unit 114, and a memory device 115.

The information acquisition unit 110 acquires human body sensor information and environmental sensor information with timing set in advance. The acquired human body sensor information and environmental sensor information are supplied to the sleep state determination unit 111 and the learning unit 112.

The sleep state determination unit 111 determines, based on the human body sensor information acquired by the information acquisition unit 110, whether the sleep state of the human body is any one of “hypnagogic”, “sleeping”, or “Wakefulness”. The determination result is supplied to the learning unit 112. The sleep state “hypnagogic” indicates a state where the human body is in the transitional state into sleep, that is, a state immediately before the human body falls asleep. The sleep state “sleeping” indicates a state where the human body is asleep. The sleep state “wakefulness” indicates a state where the human body is in the transitional state from sleep to being awake.

The learning unit 112 learns a predicted sleep rhythm based on human body sensor information and environmental sensor information, which are acquired by the information acquisition unit 110, and the determination result of the sleep state, which is obtained by the sleep state determination unit 111. The learning unit 112 also learns the influence of the operation state of the environment control apparatus 40 on sleep based on operation information indicating the operation state of the environment control apparatus 40, human body sensor information and environmental sensor information at that point in time, and the determination result of the sleep state, which is obtained by the sleep state determination unit 111. For example, the learning unit 112 learns the relationship between the operation state of the environment control apparatus 40 and the sleep state of the human body, such as a relationship where the operation of the environment control apparatus 40 to increase room temperature causes the human body to turn over, or a relationship where the operation of the environment control apparatus 40 to decrease room temperature does not cause the human body to turn over.

The control content determination unit 113 determines the control content for the environment control apparatus 40 such that the predicted sleep rhythm learned by the learning unit 112 approaches an ideal sleep rhythm stored in the memory device 115 (hereinafter referred to as “ideal sleep rhythm”). Specifically, based on the influence of the operation state of the environment control apparatus 40 on sleep, the influence being learned by the learning unit 112, the control content determination unit 113 determines the control content for the environment control apparatus 40 such that the predicted sleep rhythm approaches the ideal sleep rhythm.

Based on the control content determined by the control content determination unit 113, the apparatus control unit 114 generates a control signal to control the environment control apparatus 40. The memory device 115 stores the predicted sleep rhythm as the learning result from the learning unit 112. The memory device 115 also stores the influence of the operation state of the environment control apparatus 40 on sleep, the influence being learned by the learning unit 112. The ideal sleep rhythm used by the control content determination unit 113 is stored in advance in the memory device 115.

The controller 11 is a central processing unit (CPU), a processor, or a microcomputer that executes a program stored in the memory device 115. The memory device 115 is a nonvolatile or volatile memory, such as a random access memory (RAM), a read only memory (ROM), or a flash memory.

The information acquisition unit 110, the sleep state determination unit 111, the learning unit 112, the control content determination unit 113 and the apparatus control unit 114 of the controller 11 are function units that are achieved by the controller 11 executing a program, such as an environment improvement algorithm. The controller 11 is not limited to the above, and may be dedicated hardware that uses an application specific integrated circuit (ASIC) or a field-programmable gate array (FPGA), and the respective function units may be achieved by separate hardware or by one hardware.

### [Action of Environment Control System 100]

The action of the environment control system 100 will be described. Before describing the action of the environment control system 100, sleep rhythm will first be described.

### (Sleep Rhythm)

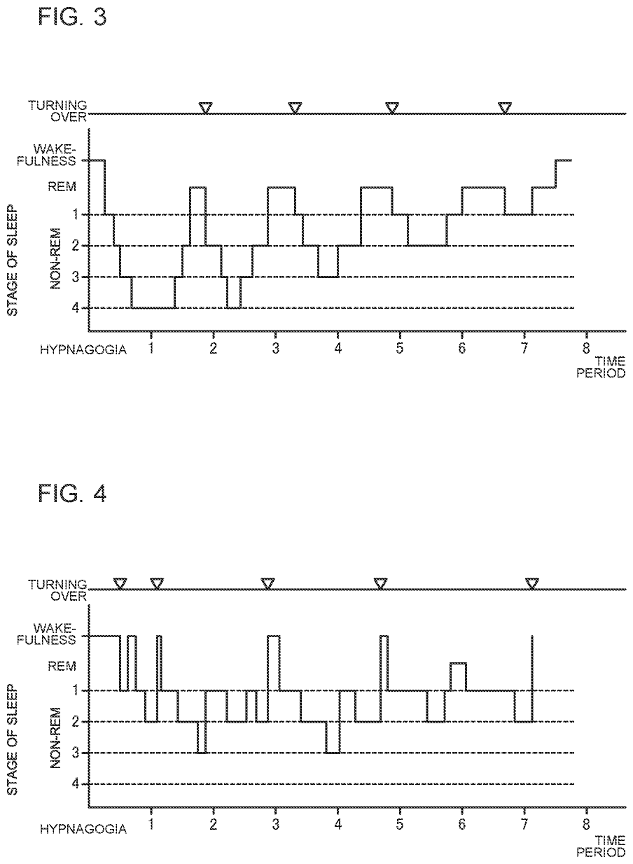

FIG. 3 is a schematic view showing one example of an ideal sleep rhythm. FIG. 4 is a schematic view showing one example of a bad sleep rhythm. In general, REM sleep, which is shallow sleep, and non-REM sleep, which is deep sleep, are repeated during sleep. Non-REM sleep has a plurality of stages of sleep state.

As shown in FIG. 3, in the case of an ideal sleep rhythm, non-REM sleep and REM sleep are repeated in approximately 90-minute cycles. In the initial stage of sleep, non-REM sleep is deep and appears for a long period of time, and non-REM sleep gradually becomes shallower and shorter toward wakefulness. At the point of time when sleep shifts to REM sleep, the human body turns over, and turning over acts as a trigger to cause sleep to shift to non-REM sleep.

In contrast, as shown in FIG. 4, in bad sleep rhythm, the cycles of non-REM sleep and REM sleep are not constant, and deep non-REM sleep does not appear. Further, the human body frequently turns over during sleep unnecessarily and hence, despite the human body being in a sleeping state, the human body is easily brought into a wakefulness state.

As described above, when sleep has a bad sleep rhythm, quality of sleep is lowered and hence, daily life or social life may be interfered with. In view of the above, in Embodiment 1, the environmental state in the room is adjusted such that the sleep rhythm of the human body is caused to approach the ideal sleep rhythm to enhance quality of sleep.

### (Schematic Action)

In the environment control system 100 according to Embodiment 1, at the time of causing the predicted sleep rhythm obtained by learning to approach the ideal sleep rhythm, the environment control apparatus 40 is controlled corresponding to the sleep state of the human body and the influence of the operation state of the environment control apparatus 40 on sleep, the influence being obtained by learning, so that the environmental state in the room is adjusted.

For example, in the case where the sleep state of the human body is a “hypnagogic” state, it is necessary to transfer heat to reduce the deep body temperature of the human body. Therefore, the controller 11, by taking into account the influence of the operation state of the environment control apparatus 40 on sleep, controls the action of the environment control apparatus 40 such that heat transfer from the human body is promoted.

In the case where the sleep state of the human body is a “sleeping” state, the controller 11, by taking into account the influence of the operation state of the environment control apparatus 40 on sleep, controls the action of the environment control apparatus 40 such that the temperature in bed is appropriately maintained by the human body turning over, and the burden on the shoulder, the waist and the like is reduced. At this point of operation, the environment control apparatus 40 may be activated to induce the human body to sleep on the side during sleep to prevent sleep apnea syndrome or to prevent snoring.

In the case where the sleep state of the human body is a “wakefulness” state, to appropriately wake the human body, the controller 11, by taking into account the influence of the operation state of the environment control apparatus 40 on sleep, controls the action of the environment control apparatus 40 such that a temperature and illuminance are gradually increased toward a scheduled waking time.

### (Sleep Environment Improving Processing)

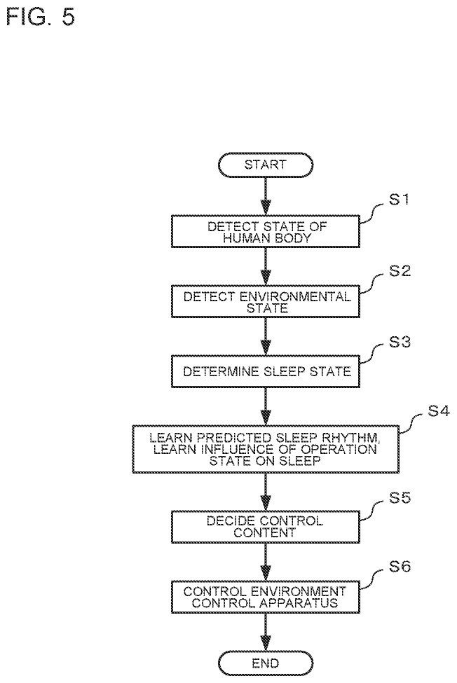

Next, a method for controlling a sleep environment with the environment control system 100 will be described. In the environment control system 100 according to Embodiment 1, sleep environment improving processing is performed to control a sleep environment. FIG. 5 is a flowchart showing one example of the flow of the sleep environment improving processing performed by the environment control system 100 according to Embodiment 1.

First, in step S1, the human body state detection unit 20 detects the state of the human body during sleep, and outputs human body sensor information. In step S2, the environmental state detection unit 30 detects the environmental state in the room, and outputs environmental sensor information. The processing in step S1 and the processing in step S2 are not limited to such an example, and may be performed in the opposite order, or may be performed simultaneously.

In step S3, the sleep state determination unit 111 of the controller 11 determines, based on the human body sensor information, whether the sleep state of the human body is any one of “hypnagogic”, “sleeping”, or “wakefulness”. In step S4, the learning unit 112 learns a predicted sleep rhythm based on the human body sensor information and the environmental sensor information, which are acquired in step S1 and step S2, and the determination result of the sleep state obtained by the sleep state determination unit 111. The learning unit 112 also learns the influence of the operation state of the environment control apparatus 40 on sleep based on the operation information of the environment control apparatus 40, the human body sensor information, the environmental sensor information, and the determination result of the sleep state obtained by the sleep state determination unit 111.

In step S5, based on the sleep state of the human body, which is determined in step S3, the predicted sleep rhythm obtained by learning in step S4, and the influence of the operation state of the environment control apparatus 40 on sleep, the control content determination unit 113 determines the control content for the environment control apparatus 40 such that the predicted sleep rhythm assumes an ideal sleep rhythm.

Specifically, when it is determined that the sleep state of the human body is “hypnagogic”, the control content determination unit 113, by taking into account the influence of the operation state of the environment control apparatus 40 on sleep, determines the control content of the environment control apparatus 40 such that heat transfer from the human body is promoted to reduce deep body temperature, so that an appropriate hypnagogic environment is provided. When it is determined that the sleep state of the human body is “sleeping”, the control content determination unit 113 further classifies the sleeping state into REM sleep and non-REM sleep. The control content determination unit 113, by taking into account the influence of the operation state of the environment control apparatus 40 on sleep, determines the control content of the environment control apparatus 40 corresponding to the classification result such that the sleep rhythm approaches the ideal sleep rhythm. When it is determined that the sleep state of the human body is “wakefulness”, the control content determination unit 113, by taking into account the influence of the operation state of the environment control apparatus 40 on sleep, determines the control content of the environment control apparatus 40 such that the human body wakes at a scheduled wake-up time.

In step S6, the apparatus control unit 114 generates a control signal for controlling the action of the environment control apparatus 40 based on the control content determined in step S5. The generated control signal is supplied to the environment control apparatus 40.

As described above, in the environment control system 100 according to Embodiment 1, based on human body sensor information and environmental sensor information, the environment control apparatus 40 is controlled such that the predicted sleep rhythm learned by the learning unit 112 approaches the ideal sleep rhythm. Therefore, it is possible to achieve a sleep environment where rhythm during sleep assumes an appropriate sleep rhythm.

In the environment control system 100, in the case where the sleep state is a hypnagogic state, the control content of the environment control apparatus is determined such that heat transfer from the human body is promoted. In the case where the sleep state is a sleeping state, the control content of the environment control apparatus is determined such that the sleep rhythm approaches the ideal sleep rhythm. In the case where the sleep state is wakefulness state, the control content of the environment control apparatus is determined such that the human body wakes at a scheduled wake-up time. With such operations, it is possible to achieve an appropriate environmental state corresponding to a sleep state.

### Embodiment 2

Next, Embodiment 2 will be described. In Embodiment 2, the case where an air-conditioning device is used as the environment control apparatus 40 will be described. In Embodiment 2, components same as the corresponding components in Embodiment 1 are given the same reference symbols, and the detailed description will be omitted.

### [Configuration of Environment Control System 200]

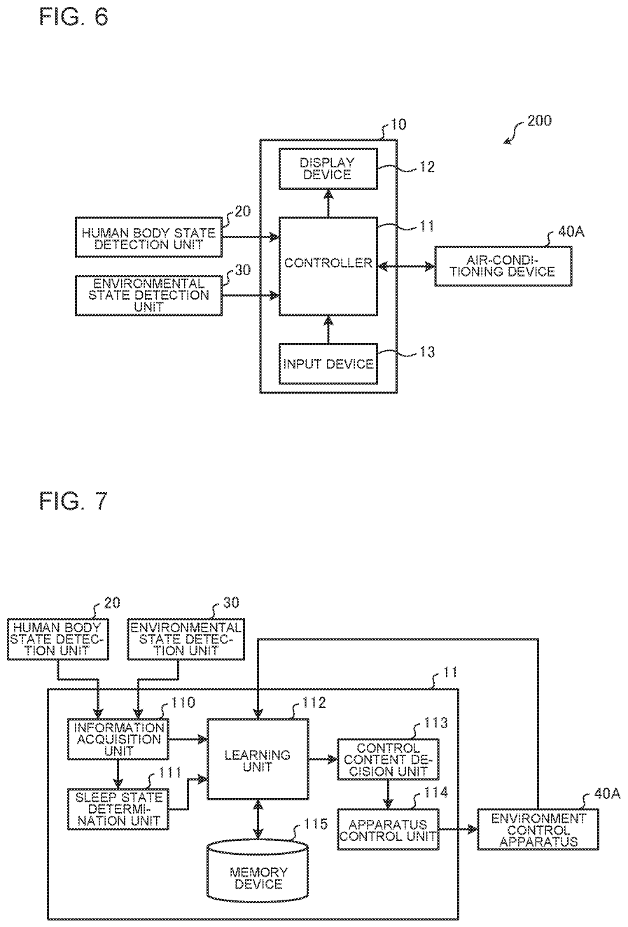

FIG. 6 is a hardware configuration diagram showing one example of the configuration of an environment control system 200 according to Embodiment 2. As shown in FIG. 6, the environment control system 200 includes the management device 10, the human body state detection unit 20, the environmental state detection unit 30, and an air-conditioning device 40A serving as the environment control apparatus.

The air-conditioning device 40A is provided for adjusting a temperature in the room. The air-conditioning device 40A includes, for example, a temperature sensor that detects an indoor temperature. The air-conditioning device 40A is activated such that the indoor temperature detected by the temperature sensor assumes a set temperature set by the user or the like. The air-conditioning device 40A can also change a wind volume, a wind direction, and a wind speed in addition to a temperature.

In the case where a temperature in the room is detected by the environmental state detection unit 30, the temperature sensor provided to the air-conditioning device 40A may be used as the environmental state detection unit 30. With such a configuration, it is unnecessary to separately provide a temperature sensor serving as the environmental state detection unit 30 and hence, the cost of equipment can be reduced.

FIG. 7 is a block diagram showing one example of the configuration of the controller 11 shown in FIG. 6. As shown in FIG. 7, the controller 11 includes the information acquisition unit 110, the sleep state determination unit 111, the learning unit 112, the control content determination unit 113, the apparatus control unit 114, and the memory device 115.

The control content determination unit 113 determines control content for the air-conditioning device 40A such that the predicted sleep rhythm learned by the learning unit 112 approaches an ideal sleep rhythm stored in the memory device 115. The apparatus control unit 114 generates, based on the control content determined by the control content determination unit 113, a control signal to control the air-conditioning device 40A.

### [Action of Environment Control System 200]

The action of the environment control system 200 will be described. In the environment control system 200 according to Embodiment 2, at the time of causing the predicted sleep rhythm obtained by learning to approach the ideal sleep rhythm, the air-conditioning device 40A is controlled corresponding to the sleep state of the human body. At this point of operation, the controller 11 adjusts a temperature, as the environmental state in the room, by taking into account the influence of the operation state of the air-conditioning device 40A on sleep, the influence being obtained by learning.

When the sleep state determination unit 111 of the controller 11 determines that the sleep state of the human body is a “hypnagogic” state, the control content determination unit 113, by taking into account the influence of the operation state of the air-conditioning device 40A on sleep, determines the control content of the air-conditioning device 40A such that an appropriate hypnagogic environment is provided, so that the predicted sleep rhythm obtained by the learning unit 112 approaches the ideal sleep rhythm. Specifically, for example, the control content determination unit 113 determines control content for the air-conditioning device 40A such that an indoor temperature falls within a set range of approximately 16 degrees C. to 26 degrees C., which is considered as an appropriate hypnagogic environment.

The control content determination unit 113 may determine control content such that a wind direction, a wind volume, a wind speed, and the like are controlled in addition to an indoor temperature. With such an operation, heat transfer from the human body in a hypnagogic state is promoted and hence, a temperature environment in bed is appropriately maintained. Therefore, it is possible to appropriately bring the human body into a hypnagogic state.

When the sleep state determination unit 111 determines that the sleep state of the human body is a “sleeping” state, the control content determination unit 113, by taking into account the influence of the operation state of the air-conditioning device 40A on sleep, determines the control content of the air-conditioning device 40A such that an appropriate sleep environment is provided, so that the predicted sleep rhythm approaches the ideal sleep rhythm. Specifically, for example, the control content determination unit 113 determines control content for the air-conditioning device 40A such that turning over is promoted. With such an operation, a temperature environment in bed during sleep is appropriately maintained, thus enhancing quality of sleep and hence, it is possible to cause the sleep rhythm of the human body to approach the ideal sleep rhythm.

When the sleep state determination unit 111 determines that the sleep state of the human body is a “wakefulness” state, the control content determination unit 113, by taking into account the influence of the operation state of the air-conditioning device 40A on sleep, determines the control content of the air-conditioning device 40A such that an appropriate wakefulness environment is provided, so that the predicted sleep rhythm approaches the ideal sleep rhythm. Specifically, for example, the control content determination unit 113 determines control content for the air-conditioning device 40A such that an indoor temperature gradually increases toward the scheduled waking time. With such an operation, wakefulness is promoted and hence, it is possible to appropriately wake the human body.

As described above, in the environment control system 200 according to Embodiment 2, the air-conditioning device 40A is used as the environment control apparatus 40. In the case where the sleep state is a hypnagogic state, the control content determination unit 113 determines the control content of the air-conditioning device 40A such that an indoor temperature falls within the set range. In the case where the sleep state is a sleeping state, the control content determination unit 113 determines the control content of the air-conditioning device 40A such that the predicted sleep rhythm approaches the ideal sleep rhythm. In the case where the sleep state is a wakefulness state, the control content determination unit 113 determines the control content of the air-conditioning device 40A such that an indoor temperature gradually increases. With such operations, it is possible to achieve an appropriate temperature state corresponding to a sleep state.

In the environment control system 200, the temperature sensor included in the air-conditioning device 40A may be used as the environmental state detection unit 30. With such a configuration, it is unnecessary to separately provide a temperature sensor serving as the environmental state detection unit 30 and hence, the cost of equipment can be reduced.

### Embodiment 3

Next, Embodiment 3 will be described. In Embodiment 3, the case where an air quality control device is used as the environment control apparatus 40 will be described. In Embodiment 3, components same as the corresponding components in Embodiments 1 and 2 are given the same reference symbols, and the detailed description will be omitted.

### [Configuration of Environment Control System 300]

FIG. 8 is a hardware configuration diagram showing one example of the configuration of an environment control system 300 according to Embodiment 3. As shown in FIG. 8, the environment control system 300 includes the management device 10, the human body state detection unit 20, the environmental state detection unit 30, and an air quality control device 40B serving as the environment control apparatus.

The air quality control device 40B is provided for adjusting humidity and air quality in the room, and has a humidity adjustment function of adjusting humidity in the room and an air quality adjustment function of removing carbon dioxide, pollen, airborne bacteria, and the like in indoor air by air circulation. For example, an air cleaner is used as the air quality control device 40B. The air quality control device 40B includes an air quality sensor that detects humidity and air quality in the room, for example. The air quality control device 40B is activated such that humidity in the room, which is detected by the air quality sensor, assumes set humidity set by the user or the like, and air quality in the room is improved.

In the case where air quality in the room is detected by the environmental state detection unit 30, the air quality sensor provided to the air quality control device 40B may be used as the environmental state detection unit 30. With such a configuration, it is unnecessary to separately provide an air quality sensor serving as the environmental state detection unit 30 and hence, the cost of equipment can be reduced.

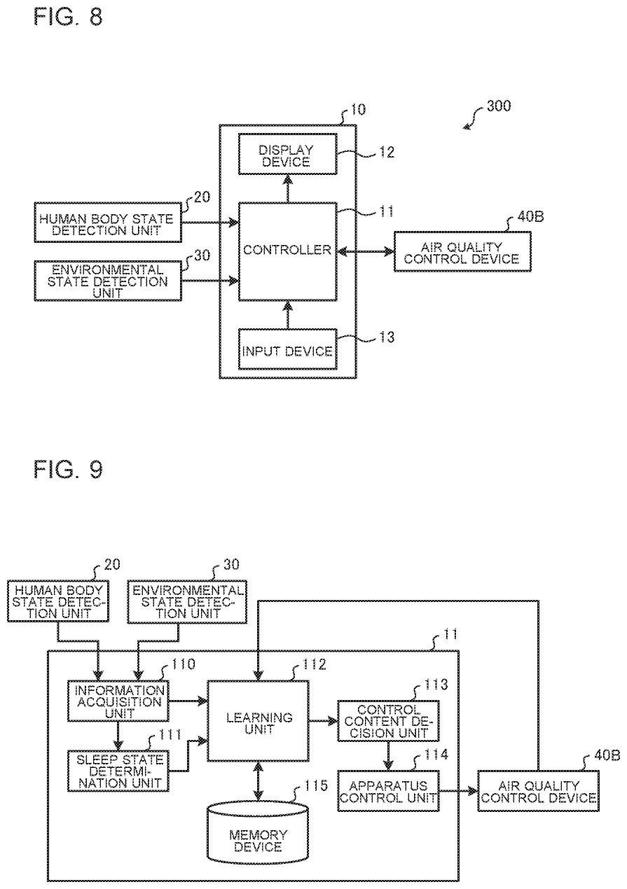

FIG. 9 is a block diagram showing one example of the configuration of the controller 11 shown in FIG. 8. As shown in FIG. 9, the controller 11 includes the information acquisition unit 110, the sleep state determination unit 111, the learning unit 112, the control content determination unit 113, the apparatus control unit 114, and the memory device 115.

The control content determination unit 113 determines control content for the air quality control device 40B such that the predicted sleep rhythm learned by the learning unit 112 approaches an ideal sleep rhythm stored in the memory device 115. The apparatus control unit 114 generates, based on the control content determined by the control content determination unit 113, a control signal to control the air quality control device 40B.

### [Action of Environment Control System 300]

The action of the environment control system 300 will be described. In the environment control system 300 according to Embodiment 3, at the time of causing the predicted sleep rhythm obtained by learning to approach the ideal sleep rhythm, the air quality control device 40B is controlled corresponding to the sleep state of the human body. At this point of operation, the controller 11 adjusts humidity and air quality, as the environmental state in the room, by taking into account the influence of the operation state of the air quality control device 40B on sleep, the influence being obtained by learning.

When the sleep state determination unit 111 of the controller 11 determines that the sleep state of the human body is a “hypnagogic” state, the control content determination unit 113, by taking into account the influence of the operation state of the air quality control device 40B on sleep, determines the control content of the air quality control device 40B such that an appropriate hypnagogic environment is provided, so that the predicted sleep rhythm obtained by the learning unit 112 approaches the ideal sleep rhythm. Specifically, for example, the control content determination unit 113 determines control content for the air quality control device 40B such that humidity in the room falls within a set range of approximately 40% to 50%, which is considered as an appropriate hypnagogic environment.

The control content determination unit 113 determines control content such that, in addition to the adjustment of humidity, air circulation is performed, so that air quality is adjusted. With such operations, a humidity environment in bed and air quality in the room are appropriately maintained and hence, it is possible to appropriately bring the human body into a hypnagogic state.

When the sleep state determination unit 111 determines that the sleep state of the human body is a “sleeping” state, the control content determination unit 113, by taking into account the influence of the operation state of the air quality control device 40B on sleep, determines the control content of the air quality control device 40B such that an appropriate sleep environment is provided, so that the predicted sleep rhythm approaches the ideal sleep rhythm. Specifically, for example, the control content determination unit 113 determines control content for the air quality control device 40B such that turning over is promoted. With such an operation, a humidity environment in bed and air quality in the room are appropriately maintained during sleep, thus enhancing quality of sleep and hence, it is possible to cause the sleep rhythm of the human body to approach the ideal sleep rhythm.

When the sleep state determination unit 111 determines that the sleep state of the human body is a “wakefulness” state, the control content determination unit 113, by taking into account the influence of the operation state of the air quality control device 40B on sleep, determines the control content of the air quality control device 40B such that an appropriate wakefulness environment is provided, so that the predicted sleep rhythm approaches the ideal sleep rhythm. Specifically, for example, the control content determination unit 113 determines control content for the air quality control device 40B such that the humidity adjustment function is stopped, and air quality is controlled by air circulation. With such an operation, wakefulness is promoted and hence, it is possible to appropriately wake the human body.

As described above, in the environment control system 300 according to Embodiment 3, the air quality control device 40B is used as the environment control apparatus 40.

In the case where the sleep state is a hypnagogic state, the control content determination unit 113 determines the control content of the air quality control device 40B such that humidity in the room falls within a set range. In the case where the sleep state is a sleeping state, the control content determination unit 113 determines the control content of the air quality control device 40B such that the predicted sleep rhythm approaches the ideal sleep rhythm. In the case where the sleep state is a wakefulness state, the control content determination unit 113 determines the control content of the air quality control device 40B such that the humidity adjustment function is stopped, and air quality is improved by air circulation. With such operations, it is possible to achieve an appropriate air quality state corresponding to a sleep state.

In the environment control system 300, the air quality sensor included in the air quality control device 40B may be used as the environmental state detection unit 30. With such a configuration, it is unnecessary to separately provide an air quality sensor serving as the environmental state detection unit 30 and hence, the cost of equipment can be reduced.

### Embodiment 4

Next, Embodiment 4 will be described. In Embodiment 4, the case where a lighting apparatus is used as the environment control apparatus 40 will be described. In Embodiment 4, components same as the corresponding components in Embodiments 1 to 3 are given the same reference symbols, and the detailed description will be omitted.

### [Configuration of Environment Control System 400]

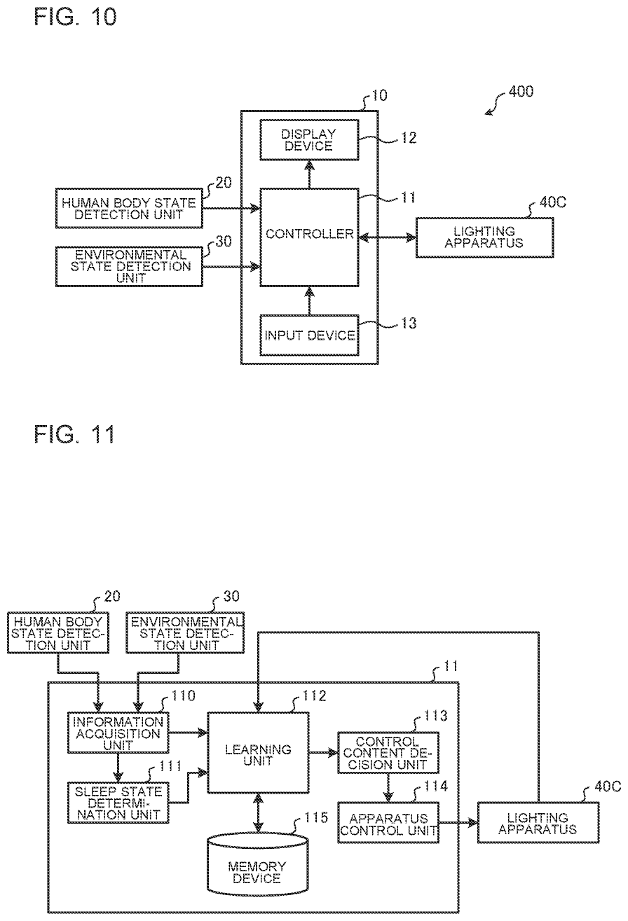

FIG. 10 is a hardware configuration diagram showing one example of the configuration of an environment control system 400 according to Embodiment 4. As shown in FIG. 10, the environment control system 400 includes the management device 10, the human body state detection unit 20, the environmental state detection unit 30, and a lighting apparatus 40C serving as the environment control apparatus.

The lighting apparatus 40C is provided for adjusting illuminance in the room, for example. The lighting apparatus 40C includes, for example, an illuminance sensor that detects illuminance in the room. The lighting apparatus 40C is activated such that the illuminance in the room, which is detected by the illuminance sensor, assumes a set illuminance set by the user or the like.

In the case where illuminance in the room is detected by the environmental state detection unit 30, the illuminance sensor provided to the lighting apparatus 40C may be used as the environmental state detection unit 30. With such a configuration, it is unnecessary to separately provide an illuminance sensor serving as the environmental state detection unit 30 and hence, the cost of equipment can be reduced.

FIG. 11 is a block diagram showing one example of the configuration of the controller 11 shown in FIG. 10. As shown in FIG. 11, the controller 11 includes the information acquisition unit 110, the sleep state determination unit 111, the learning unit 112, the control content determination unit 113, the apparatus control unit 114, and the memory device 115.

The control content determination unit 113 determines control content for the lighting apparatus 40C such that the predicted sleep rhythm learned by the learning unit 112 approaches an ideal sleep rhythm stored in the memory device 115. The apparatus control unit 114 generates, based on the control content determined by the control content determination unit 113, a control signal to control the lighting apparatus 40C.

### [Action of Environment Control System 400]

The action of the environment control system 400 will be described. In the environment control system 400 according to Embodiment 4, at the time of causing the predicted sleep rhythm obtained by learning to approach the ideal sleep rhythm, the lighting apparatus 40C is controlled corresponding to the sleep state of the human body. At this point of operation, the controller 11 adjusts illuminance, as the environmental state in the room, by taking into account the influence of the operation state of the lighting apparatus 40C on sleep, the influence being obtained by learning.

When the sleep state determination unit 111 of the controller 11 determines that the sleep state of the human body is a “hypnagogic” state, the control content determination unit 113, by taking into account the influence of the operation state of the lighting apparatus 40C on sleep, determines the control content of the lighting apparatus 40C such that an appropriate hypnagogic environment is provided, so that the predicted sleep rhythm obtained by the learning unit 112 approaches the ideal sleep rhythm. Specifically, for example, the control content determination unit 113 determines control content for the lighting apparatus 40C such that illuminance decreases in a stepwise manner. With such an operation, illuminance in the room is appropriately set and hence, it is possible to appropriately bring the human body into a hypnagogic state.

When the sleep state determination unit 111 determines that the sleep state of the human body is a “sleeping” state, the control content determination unit 113, by taking into account the influence of the operation state of the lighting apparatus 40C on sleep, determines the control content of the lighting apparatus 40C such that an appropriate sleep environment is provided, so that the predicted sleep rhythm approaches the ideal sleep rhythm. Specifically, for example, the control content determination unit 113 determines control content for the lighting apparatus 40C such that illuminance assumes illuminance suitable for sleep. With such an operation, air quality in the room is appropriately set during sleep, thus enhancing quality of sleep and hence, it is possible to cause the sleep rhythm of the human body to approach the ideal sleep rhythm.

When the sleep state determination unit 111 determines that the sleep state of the human body is a “wakefulness” state, the control content determination unit 113, by taking into account the influence of the operation state of the lighting apparatus 40C on sleep, determines the control content of the lighting apparatus 40C such that an appropriate wakefulness environment is provided, so that the predicted sleep rhythm approaches the ideal sleep rhythm. Specifically, for example, the control content determination unit 113 determines control content for the lighting apparatus 40C such that an indoor temperature gradually increases toward the scheduled waking time. With such an operation, wakefulness is promoted and hence, it is possible to appropriately wake the human body.

As described above, in the environment control system 400 according to Embodiment 4, the lighting apparatus 40C is used as the environment control apparatus 40.

In the case where the sleep state is a hypnagogic state, the control content determination unit 113 determines the control content of the lighting apparatus 40C such that illuminance in the room decreases in a stepwise manner. In the case where the sleep state is a sleeping state, the control content determination unit 113 determines the control content of the lighting apparatus 40C such that illuminance in the room assumes illuminance suitable for sleep. In the case where the sleep state is a wakefulness state, the control content determination unit 113 determines the control content of the lighting apparatus 40C such that illuminance in the room gradually increases. With such operations, it is possible to achieve an appropriate illuminance state corresponding to a sleep state.

In the environment control system 400, the illuminance sensor included in the lighting apparatus 40C may be used as the environmental state detection unit 30. With such a configuration, it is unnecessary to separately provide an illuminance sensor serving as the environmental state detection unit 30 and hence, the cost of equipment can be reduced.

### Embodiment 5

Next, Embodiment 5 will be described. In Embodiment 5, the case where an audio apparatus is used as the environment control apparatus 40 will be described. In Embodiment 5, components same as the corresponding components in Embodiments 1 to 4 are given the same reference symbols, and the detailed description will be omitted.

### [Configuration of Environment Control System 500]

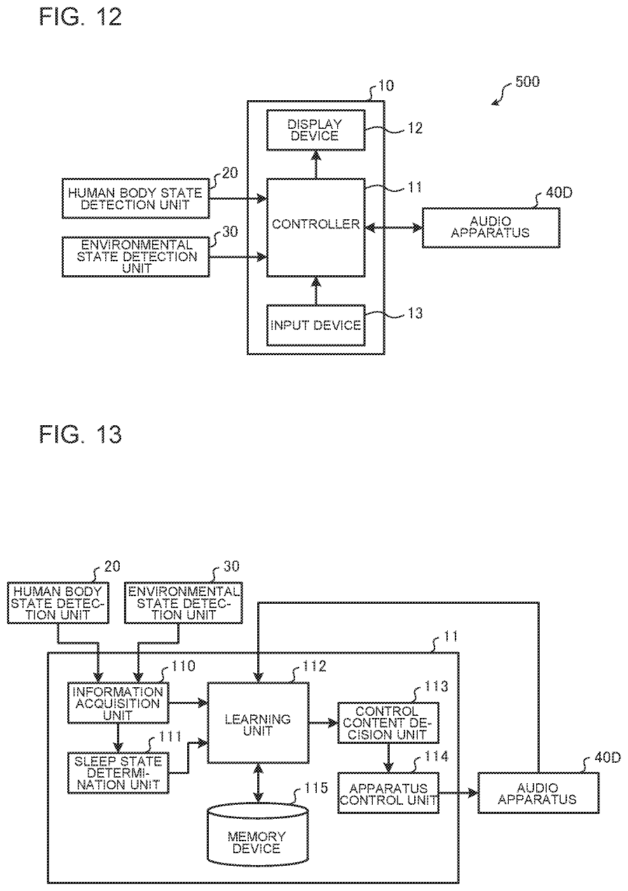

FIG. 12 is a hardware configuration diagram showing one example of the configuration of an environment control system 500 according to Embodiment 5. As shown in FIG. 12, the environment control system 500 includes the management device 10, the human body state detection unit 20, the environmental state detection unit 30, and an audio apparatus 40D serving as the environment control apparatus.

The audio apparatus 40D is provided for adjusting the acoustics in the room. The audio apparatus 40D can output, in addition to the sound of music or the like, insect chirping, or the sound of various kinds of noise, such as white noise, pink noise or brown noise. The audio apparatus 40D is activated such that sound and volume assume sound and volume set by the user or the like.

FIG. 13 is a block diagram showing one example of the configuration of the controller 11 shown in FIG. 12. As shown in FIG. 11, the controller 11 includes the information acquisition unit 110, the sleep state determination unit 111, the learning unit 112, the control content determination unit 113, the apparatus control unit 114, and the memory device 115.

The control content determination unit 113 determines control content for the audio apparatus 40D such that the predicted sleep rhythm learned by the learning unit 112 approaches an ideal sleep rhythm stored in the memory device 115. The apparatus control unit 114 generates, based on the control content determined by the control content determination unit 113, a control signal to control the audio apparatus 40D.

### [Action of Environment Control System 500]

The action of the environment control system 500 will be described. In the environment control system 500 according to Embodiment 5, at the time of causing the predicted sleep rhythm obtained by learning to approach the ideal sleep rhythm, the audio apparatus 40D is controlled corresponding to the sleep state of the human body. At this point of operation, the controller 11 adjusts sound and volume, as the environmental state in the room, by taking into account the influence of the operation state of the audio apparatus 40D on sleep, the influence being obtained by learning.

When the sleep state determination unit 111 of the controller 11 determines that the sleep state of the human body is a “hypnagogic” state, the control content determination unit 113, by taking into account the influence of the operation state of the audio apparatus 40D on sleep, determines the control content of the audio apparatus 40D such that an appropriate hypnagogic environment is provided, so that the predicted sleep rhythm obtained by the learning unit 112 approaches the ideal sleep rhythm. Specifically, for example, the control content determination unit 113 determines control content for the audio apparatus 40D such that sound and volume assume sound and volume suitable for a hypnagogic environment. With such an operation, sound and volume in the room are appropriately set and hence, it is possible to appropriately bring the human body into a hypnagogic state.

When the sleep state determination unit 111 determines that the sleep state of the human body is a “sleeping” state, the control content determination unit 113, by taking into account the influence of the operation state of the audio apparatus 40D on sleep, determines the control content of the audio apparatus 40D such that an appropriate sleep environment is provided, so that the predicted sleep rhythm approaches the ideal sleep rhythm. Specifically, for example, the control content determination unit 113 determines control content for the audio apparatus 40D such that the sound and volume of the audio assume sound and volume suitable for sleep. With such an operation, the acoustics in the room are appropriately set during sleep, thus enhancing quality of sleep and hence, it is possible to cause the sleep rhythm of the human body to approach the ideal sleep rhythm.

When the sleep state determination unit 111 determines that the sleep state of the human body is a “wakefulness” state, the control content determination unit 113, by taking into account the influence of the operation state of the audio apparatus 40D on sleep, determines the control content of the audio apparatus 40D such that an appropriate wakefulness environment is provided, so that the predicted sleep rhythm approaches the ideal sleep rhythm. Specifically, for example, the control content determination unit 113 determines control content for the audio apparatus 40D such that volume gradually increase toward the scheduled waking time. With such an operation, wakefulness is promoted and hence, it is possible to appropriately wake the human body.

As described above, in the environment control system 500 according to Embodiment 5, the audio apparatus 40D is used as the environment control apparatus 40. In the case where the sleep state is a hypnagogic state, the control content determination unit 113 determines the control content of the audio apparatus 40D such that sound and volume in the room assume sound and volume suitable for a hypnagogic environment. In the case where the sleep state is a sleeping state, the control content determination unit 113 determines the control content of the audio apparatus 40D such that sound and volume in the room assume sound and volume suitable for sleep. In the case where the sleep state is a wakefulness state, the control content determination unit 113 determines the control content of the audio apparatus 40D such that volume gradually increases. With such operations, it is possible to achieve an appropriate audio state corresponding to a sleep state.

### Embodiment 6

Next, Embodiment 6 will be described. In Embodiment 6, the case where an aroma generating device is used as the environment control apparatus 40 will be described. In Embodiment 6, components same as the corresponding components in Embodiments 1 to 5 are given the same reference symbols, and the detailed description will be omitted.

### [Configuration of Environment Control System 600]

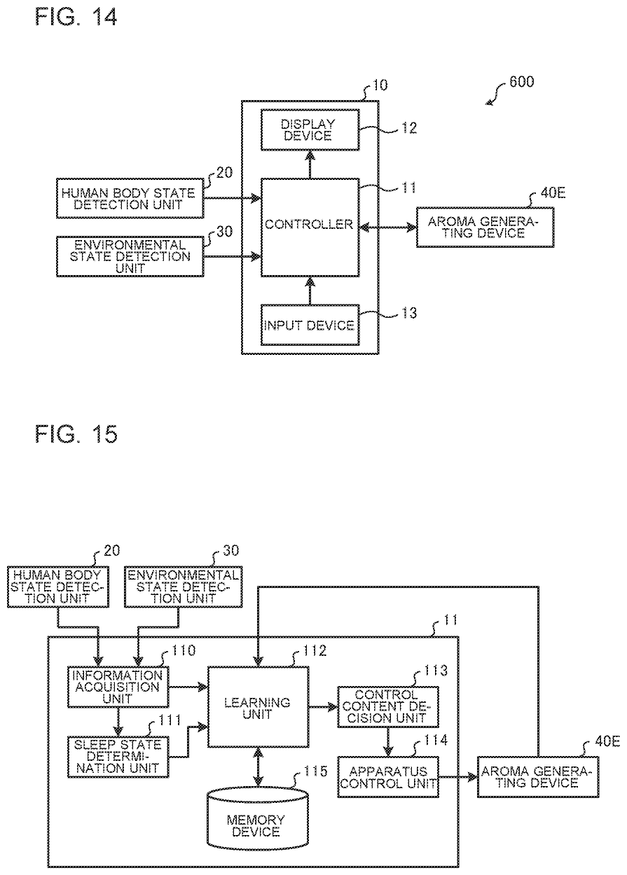

FIG. 14 is a hardware configuration diagram showing one example of the configuration of an environment control system 600 according to Embodiment 6. As shown in FIG. 14, the environment control system 600 includes the management device 10, the human body state detection unit 20, the environmental state detection unit 30, and an aroma generating device 40E serving as the environment control apparatus.

The aroma generating device 40E is provided for adjusting scent in the room. The aroma generating device 40E can output a plurality of kinds of scent suitable for a sleep state, such as a lavender scent or a citrus scent. The aroma generating device 40E is activated such that the kind and intensity of scent assume the kind and intensity of scent set by the user or the like.

FIG. 15 is a block diagram showing one example of the configuration of the controller 11 shown in FIG. 14. As shown in FIG. 15, the controller 11 includes the information acquisition unit 110, the sleep state determination unit 111, the learning unit 112, the control content determination unit 113, the apparatus control unit 114, and the memory device 115.

The control content determination unit 113 determines control content for the aroma generating device 40E such that the predicted sleep rhythm learned by the learning unit 112 approaches an ideal sleep rhythm stored in the memory device 115. The apparatus control unit 114 generates, based on the control content determined by the control content determination unit 113, a control signal to control the aroma generating device 40E.

### [Action of Environment Control System 600]

The action of the environment control system 600 will be described. In the environment control system 600 according to Embodiment 6, at the time of causing the predicted sleep rhythm obtained by learning to approach the ideal sleep rhythm, the aroma generating device 40E is controlled corresponding to the sleep state of the human body. At this point of operation, the controller 11 adjusts the kind and intensity of scent, as the environmental state in the room, by taking into account the influence of the operation state of the aroma generating device 40E on sleep, the influence being obtained by learning.

When the sleep state determination unit 111 of the controller 11 determines that the sleep state of the human body is a “hypnagogic” state, the control content determination unit 113, by taking into account the influence of the operation state of the aroma generating device 40E on sleep, determines control content for the aroma generating device 40E such that an appropriate hypnagogic environment is provided, so that the predicted sleep rhythm obtained by the learning unit 112 approaches the ideal sleep rhythm. Specifically, for example, the control content determination unit 113 determines control content for the aroma generating device 40E such that the kind and intensity of scent in the room assume the kind and intensity of scent suitable for a hypnagogic environment. With such an operation, the kind and intensity of scent in the room are appropriately set and hence, it is possible to appropriately bring the human body into a hypnagogic state.

When the sleep state determination unit 111 determines that the sleep state of the human body is a “sleeping” state, the control content determination unit 113, by taking into account the influence of the operation state of the aroma generating device 40E on sleep, determines control content for the aroma generating device 40E such that an appropriate sleep environment is provided, so that the predicted sleep rhythm approaches the ideal sleep rhythm. Specifically, for example, the control content determination unit 113 determines control content for the aroma generating device 40E such that the kind and intensity of scent in the room assume the kind and intensity of scent suitable for sleep. With such an operation, scent in the room is appropriately set during sleep, thus enhancing quality of sleep and hence, it is possible to cause the sleep rhythm of the human body to approach the ideal sleep rhythm.

When the sleep state determination unit 111 determines that the sleep state of the human body is a “wakefulness” state, the control content determination unit 113, by taking into account the influence of the operation state of the aroma generating device 40E on sleep, determines control content for the aroma generating device 40E such that an appropriate wakefulness environment is provided, so that the predicted sleep rhythm approaches the ideal sleep rhythm. Specifically, for example, the control content determination unit 113 determines control content for the aroma generating device 40E such that the kind and intensity of scent in the room assume the kind and intensity of scent suitable for wakefulness. With such an operation, wakefulness is promoted and hence, it is possible to appropriately wake the human body.

As described above, in the environment control system 600 according to Embodiment 6, the aroma generating device 40E is used as the environment control apparatus 40. In the case where the sleep state is a hypnagogic state, the control content determination unit 113 determines the control content of the aroma generating device 40E such that the kind and intensity of scent in the room assume the kind and intensity of scent suitable for a hypnagogic environment. In the case where the sleep state is a sleeping state, the control content determination unit 113 determines the control content of the aroma generating device 40E such that the kind and intensity of scent in the room assume the kind and intensity of scent suitable for sleep. In the case where the sleep state is a wakefulness state, the control content determination unit 113 determines the control content of the aroma generating device 40E such that the kind of scent in the room assumes the kind of scent suitable for wakefulness. With such operations, it is possible to achieve an appropriate scent state corresponding to a sleep state.

Embodiments 1 to 6 have been described heretofore. However, the environment control system is not limited to the above-mentioned Embodiments 1 to 6, and various modifications and applications are conceivable without departing from the gist. For example, in each of Embodiments 1 to 6, the description has been made for a case where a different apparatus is used as the environment control apparatus 40. However, the configuration is not limited to the above. It is also possible to control the environment control apparatus 40 including two or more kinds of apparatuses selected from the respective apparatuses in Embodiments 1 to 6.

In this example, the state of the human body in the sleep state from hypnagogia to wakefulness is detected. However, the configuration is not limited to the above. It is also possible to further detect the state of the human body during activity. In this case, a predicted sleep model may be learned based on the state of the human body during activity.

It is also possible to adopt a configuration where good/bad evaluation of sleep is inputted by the user, and the input result is reflected on learning. In this case, for example, the user inputs good/bad evaluation of sleep by using the input device 13. The controller 11 of the management device 10 corrects control content during sleep based on the input result. With such an operation, at the time of causing the predicted sleep rhythm to approach the ideal sleep rhythm, it is possible to cause the environment control apparatus 40 to be activated more appropriately.

In sleep rhythm described with reference to FIG. 3 and FIG. 4, the depth of non-REM sleep has a plurality of sleep stages. Such stages of the depth of sleep cannot be detected by the normal human body state detection unit 20, which is a temperature sensor or any other sensor. Accordingly, when sleep rhythm is predicted with the learning unit 112, the stage of the depth of sleep is estimated by algorithm or the like determined in advance.

In the case where an electroencephalograph that measures brain waves of the human body, or a deep body temperature measurement device that measures the deep body temperature of the human body is used as the human body state detection unit 20, the depth of sleep during sleep can be measured. Therefore, the learning unit 112 may learn a predicted sleep rhythm by using the measurement result from such a human body state detection unit 20.

### REFERENCE SIGNS LIST

10: management device, 11: controller, 12: display device, 13: input device, 20: human body state detection unit, 30: environmental state detection unit, 40: environment control apparatus, 40A: air-conditioning device, 40B: air quality control device, 40C: lighting apparatus, 40D: audio apparatus, 40E: aroma generating device, 100, 200, 300, 400, 500, 600: environment control system, 110: information acquisition unit, 111: sleep state determination unit, 112: learning unit, 113: control content determination unit, 114: apparatus control unit, 115: memory device.

1. An environment control system comprising:
an environment control apparatus installed in a room, and configured to adjust an indoor environment;
a human body state detection unit configured to detect a state of a human body present in the room as human body sensor information;
an environmental state detection unit configured to detect an environmental state in the room as environmental sensor information; and
a controller configured to
determine, based on the human body sensor information, whether a sleep state is any one of a hypnagogic state, which is a state before the human body falls asleep, a sleeping state where the human body is asleep, or a wakefulness state where the human body is in a transitional state from sleep to being awake;
learn a predicted sleep rhythm of the human body based on the human body sensor information, the environmental sensor information, and a determination result of the sleep state;
determine control content of the environment control apparatus such that the predicted sleep rhythm obtained by learning approaches an ideal sleep rhythm; and
control the environment control apparatus based on the control content determined.

2. The environment control system of claim 1, wherein
in a case where the sleep state is the hypnagogic state, the controller determines the control content of the environment control apparatus such that heat transfer from the human body is promoted,
in a case where the sleep state is the sleeping state, the controller determines the control content of the environment control apparatus such that the predicted sleep rhythm approaches the ideal sleep rhythm, and
in a case where the sleep state is the wakefulness state, the controller determines the control content of the environment control apparatus such that the human body wakes at a scheduled wake-up time.

3. The environment control system of claim 1, wherein
the controller further learns an influence of an operation state of the environment control apparatus on sleep based on operation information indicating the operation state of the environment control apparatus, the human body sensor information, the environmental sensor information, and the determination result of the sleep state, and
the controller determines the control content of the environment control apparatus by further using the influence of the operation state of the environment control apparatus on sleep, the influence being obtained by learning.

4. The environment control system of claim 1, wherein
the environment control apparatus is an air-conditioning device that adjusts a temperature in the room,
in the case where the sleep state is the hypnagogic state, the controller determines the control content of the air-conditioning device such that an indoor temperature falls within a set range,
in the case where the sleep state is the sleeping state, the controller determines the control content of the air-conditioning device such that the predicted sleep rhythm approaches the ideal sleep rhythm, and
in the case where the sleep state is the wakefulness state, the controller determines the control content of the air-conditioning device such that the indoor temperature gradually increases.

5. The environment control system of claim 4, wherein
the air-conditioning device includes a temperature sensor configured to detect the indoor temperature, and
the temperature sensor is used as the environmental state detection unit.

6. The environment control system of claim 1, wherein
the environment control apparatus is an air quality control device that adjusts humidity and air quality in the room,
in the case where the sleep state is the hypnagogic state, the controller determines the control content of the air quality control device such that the humidity in the room falls within a set range,
in the case where the sleep state is the sleeping state, the controller determines the control content of the air quality control device such that the predicted sleep rhythm approaches the ideal sleep rhythm, and
in the case where the sleep state is the wakefulness state, the controller determines the control content of the air quality control device such that a humidity adjustment function is stopped, and air quality is improved by air circulation.

7. The environment control system of claim 6, wherein
the air quality control device includes an air quality sensor configured to detect humidity and air quality in the room, and
the air quality sensor is used as the environmental state detection unit.

8. The environment control system of claim 1, wherein
the environment control apparatus is a lighting apparatus that adjusts illuminance in the room,
in the case where the sleep state is the hypnagogic state, the controller determines the control content of the lighting apparatus such that the illuminance in the room decreases in a stepwise manner,
in the case where the sleep state is the sleeping state, the controller determines the control content of the lighting apparatus such that the illuminance in the room assumes illuminance suitable for sleep, and
in the case where the sleep state is the wakefulness state, the controller determines the control content of the lighting apparatus such that the illuminance in the room gradually increases.

9. The environment control system of claim 8, wherein
the lighting apparatus includes an illuminance sensor configured to detect illuminance in the room, and
the illuminance sensor is used as the environmental state detection unit.

10. The environment control system of claim 1, wherein
the environment control apparatus is an audio apparatus that adjusts acoustics in the room,
in the case where the sleep state is the hypnagogic state, the controller determines the control content of the audio apparatus such that sound and volume in the room assume sound and volume suitable for a hypnagogic environment,
in the case where the sleep state is the sleeping state, the controller determines the control content of the audio apparatus such that the sound and volume in the room assume sound and volume suitable for sleep, and
in the case where the sleep state is the wakefulness state, the controller determines the control content of the audio apparatus such that the volume gradually increases.

11. The environment control system of claim 1, wherein
the environment control apparatus is an aroma generating device that adjusts a kind and intensity of scent in the room,
in the case where the sleep state is the hypnagogic state, the controller determines the control content of the aroma generating device such that the kind and intensity of the scent in the room assume a kind and intensity of the scent suitable for the hypnagogic environment,
in the case where the sleep state is the sleeping state, the controller determines the control content of the aroma generating device such that the kind and intensity of the scent in the room assume a kind and intensity of the scent suitable for sleep, and
in the case where the sleep state is the wakefulness state, the controller determines the control content of the aroma generating device such that the kind and intensity of the scent in the room assume a kind and intensity of the scent suitable for wakefulness.

12. A method for controlling an environment, the method comprising:
detecting a state of a human body present in a room as human body sensor information;
detecting an environmental state in the room as environmental sensor information;
determining, based on the human body sensor information, whether a sleep state is any one of a hypnagogic state, which is a state before the human body falls asleep, a sleeping state where the human body is asleep, or a wakefulness state where the human body is in a transitional state from sleep to being awake;
learning a predicted sleep rhythm of the human body based on the human body sensor information, the environmental sensor information, and a determination result of the sleep state;
deciding control content of an environment control apparatus such that the predicted sleep rhythm obtained by learning approaches an ideal sleep rhythm, the environment control apparatus being installed in the room; and
controlling the environment control apparatus based on the control content determined.
